(19) United States
(12) Patent Application Publication
(10) Pub. No.: US 2008/0222781 A1
(43) Pub. Date: Sep. 18, 2008

(54) Undergarment for prevention of leaks and permanent stains

(76) Inventor: Kathleen K. Rhew, Brooklyn, NY (US)

Correspondence Address: MORGAN & FINNEGAN, L.L.P., 3 World Financial Center, New York, NY, 10281-2101, US

(21) Appl. No.: 11/724,659

(22) Filed: Mar. 15, 2007

(51) Int. Cl. A41B 9/04; A61M 1/00

(52) U.S. Cl. 2/406; 604/317

(57) ABSTRACT

An undergarment to be worn primarily by women during menstruation that resists staining of the undergarment itself and prevents leakage of menstrual flow to outer garments. The outer covering of the undergarment with a waist portion and a pair of leg openings is composed of fabric to make the fabric repel stains, repel and release stains, or absorb and then release stains. A panel of fabric treated to make the fabric repel stains, repel and release stains, or absorb and then release stains covers the crotch area and may extend up the front and/or back of the undergarment. Fabric on each side of the panel, also made of fabric treated to make the fabric repel stains, repel and release stains, or absorb and then release stains, provides additional protection to prevent leakage. In addition, in the preferred embodiment, the undergarment will have antimicrobial properties. This undergarment is intended to be worn in lieu of a regular undergarment and with a tampon, sanitary napkin and/or other personal hygiene device.

### FIELD OF THE INVENTION

The present invention relates to lower-body undergarments for women to prevent staining and leaking during menstruation.

### BACKGROUND OF THE INVENTION

During menstruation, women are concerned about staining their undergarments as well as leakage of menstrual flow and the subsequent staining of apparel and bedding (when sleeping) even when they are wearing a tampon, sanitary napkin, and/or other feminine hygiene device. Despite all of the improvements in feminine hygiene products including super absorbent tampons and longer, more absorbent maxipads with side flaps or “wings”, women still experience leakage and staining of their undergarments, apparel and bedding (when sleeping). Dealing with stains is frustrating and time-consuming because blood stains can be difficult to treat. In addition, when leaks occur and the staining becomes visible on clothing and/or bedsheets, women experience embarrassment. Even the possibility of such an embarrassing leakage and stain can cause women anxiety and worry.

Women sometimes inadequately attempt to deal with the problem (or the potential of this problem) of staining and leakage by wearing dark-colored underwear and dark-colored clothing so that any staining would not be readily detectable. However, doing so limits a woman's choice of underwear and clothing and does not prevent the actual staining and the discomfort from leakage.

At least one undergarment has been offered that requires that at least some part of the panty is black in color. For example, the Philpott patent (U.S. Pat. No. 5,944,708) discloses a protective menstrual panty which is comprised of a black LYCRA fabric. As mentioned, this color requirement limits a woman's choice of underwear and does not prevent the staining and discomfort from leakage.

Some undergarments have at least three layers of fibers. As can be seen in several patents (Kennedy (U.S. Pat. No. 3,613,687) and Lamb (U.S. Pat. No. 4,573,987)), one solution provides multi-layer reusable diapers and diaper-like garments. However, multiple layers of fabric feel bulky and diaper-like and therefore are inadequate to address the problem.

Other solutions provide a liquid absorbent layer next to the skin with no special treatment to release stains and a liquid repellent layer on the outside of the garment. Bonito (U.S. Pat. No. 4,718,902) and Feder (U.S. Pat. No. 6,782,557). The absorbent layer next to the skin absorbs fluids and stains, leading to permanent staining, and causing leakage to outer clothing.

Other solutions provide for waterproof layers made from plastic, non-breathable, non-stretchable, polyester or coated with a modified polyester urethane polymer. As disclosed in Branch (U.S. Pat. No. 4,813,950), Rainville-Lonn (U.S. Patent Application No. 2004/0230175), and Gold (U.S. Pat. No. 5,098,419), several undergarments have been described having layers made from plastic. However, these solutions can be uncomfortable.

Nozaki (U.S. Pat. No. 6,613,034) and Coates (patent application) describe a suspended sling with a lining pad and extension cloth for extra protection.

Women are also concerned about the odors that may accompany menstruation. Women have tried to deal with this problem by using feminine deodorants and scented pads and tampons. However, these products often have their own unique odors that are closely associated with those products, and some women do not like to smell like such products.

Accordingly, a primary object of the present invention is to provide an undergarment that uses a minimal number of layers of fabric.

Another object is to provide an undergarment that is stain repellant and/or contains stain release technology in that the material itself has stain repel, stain repel and release, and/or stain release properties.

Yet another object of the present invention is to provide an undergarment that looks natural and comfortable.

A further object of the present invention is to provide an undergarment that is made from gas permeable material, thus making it breathable.

Another object of the present invention is to provide an undergarment that is easy to use for the wearer.

Yet another object of the present invention is to provide an undergarment that is re-usable and machine washable.

A further object of the present invention is to provide an undergarment which will accept disposable panty shields or sanitary pads for menstruation.

To accomplish the above and related objects, this invention may be embodied in the form illustrated in the accompanying drawings. However, the drawings are illustrative only and changes may be made to the specific construction shown and described within the scope of the claims.

### SUMMARY OF THE INVENTION

The present invention is directed to resolution of the problem of leakage and staining of undergarments and outerwear during menstruation as well as the problem of odors from menstrual flow. Other solutions have been offered, but none provide the unique combination of benefits offered by this invention.

Fabric used in this invention will be treated with chemical enhancements to make the fabric repel (or resist) stains, repel and release stains, and/or release stains. Such fabrics are also known as fabrics with soil-and stain-resistant finishes and soil release finishes.

This undergarment is intended for women of all ages from the start of menarche through menopause. The undergarment looks and feels like a standard underwear from the exterior and can be adapted for a variety of styles including, without limitation, bikinis, boy shorts, briefs, hip huggers and low-rise. The structure of the crotch area of this undergarment contains a border, preferably in the shape of a ruffle, which folds inward and/or outward providing extra protection from leakage and staining.

### BRIEF DESCRIPTION OF THE DRAWINGS

The above and other features, advantages, and objects of the invention may become more apparent when viewed in conjunction with the accompanying drawings that depict certain preferred embodiments of the invention.

FIG. 1 is a front view of a panty constructed in accordance with the invention with a ruffle-bordered interior panel extending partially up the back of the panty and in partial phantom outline;

FIG. 2 is a rear view, with the edges from the front of the panty partly in phantom outline, of the panty shown in FIG. 1;

FIG. 3 is a folded out view of an interior panel covering the crotch area with a ruffled border and extending partially up the backside of the panty shown in FIG. 1;

FIG. 4 is a front view of a panty constructed in accordance with the invention with a ruffle-bordered interior panel extending fully up the back of the panty and in partial phantom outline;

FIG. 5 is a rear view, with edges from the front of the panty partly in phantom outline, of the panty shown in FIG. 4; and

FIG. 6 is a folded out view of an interior panel covering the crotch area with a ruffle border and extending fully up the backside of the panty shown in FIG. 4.

### DETAILED DESCRIPTION OF THE INVENTION

The present invention relates to an undergarment which is stain repellant and/or stain release. Such an undergarment is particularly suited for use by women during the menstrual cycle. In addition, other situations for men, women and children may be conducive of use of the stain repel and/or stain release undergarment of the present invention. As discussed in more detail hereinbelow, the undergarment may be of any suitable style, so long as all or part of the undergarment comprises a stain repel and/or stain release fabric. The undergarment may be single layer or multi-layer.

A variety of chemical processes are known to impart stain release and/or stain repel characteristics onto fabrics. Many such chemistries are known in the art of outerwear or carpeting but have not been used in connection with an undergarment as described herein. “Stain repel” and “stain resist” are two terms used in the art (used synonymously herein) to describe the characteristic of a fabric wherein liquids such as water and/or oils are prevented from penetrating the fabric, but instead bead up and roll off the fabric. This characteristic is referred to herein as “stain repel”. A second type of advantageous fabric treatment is a “stain release” treatment, which enhances the ability of a fabric to release stains during laundering. For a stain release finish, liquids may not bead up, but may soak into the fabric, only to be easily washed away.

One method for creating stain repel and/or stain release fabrics uses fluorochemical treatments, which can impart fabrics with stain repellent characteristics as well as stain release characteristics depending upon the particular treatment methods employed. Examples of such methods are incorporated by reference and can be found in McLarty, (U.S. Pat. No. 5,855,991), Huber (U.S. Pat. No. 5,284,902), and Dams (U.S. Pat. No. 5,276,175). Other stain repel finishing agents include: flourocarbons, fluorochemicals, pyridinum compounds, resins, silicone, triazine compounds, wax and wax-like derivatives. All of these treatment methods can be used for the panty of the invention and can be found for example in Vasquez (U.S. Pat. No. 7,097,785), Theoliere (U.S. Pat. No. 7,094,743), Bamabas (U.S. Pat. No. 7,012,053), Beck (U.S. Pat. No. 5,002,684), and Degenhardt (U.S. Pat. No. 5,009,882). These patent disclosures are incorporated herein, in toto, by reference.

Stain repellants may be used on a variety of fabrics for use in the present invention. These are chemical treatments that can be used on any preferred fabric, for example but not limited to cotton, cotton blends, bamboo, bamboo blends, synthetics (such as acetate, nylon (polyamide), olefins (polypropylene, polyethylene), polyester, and or spandex) and synthetic blends. The main advantage is that the fabrics resist soiling during use. When a leak occurs and is still fresh, it can be spot-cleaned easily, since the stain is confined to the surface rather than penetrating deep into the fabric. Such finishes may employ the use of a fluorochemical, either alone or in conjunction with an extender. Applying a stain repellent finish prevents liquids, including water and/or oils, from penetrating the fabric causing potential aqueous and oily stains to bead up and roll off. Test methods used for evaluation include Oil Repellency (AATCC 118) for oily stain resistance and the Isopropanol/Water Drop test for aqueous stain resistance.

Fluorochemical water/stain repellent finishes provide durable liquid repellence (water and oil) without compromising the natural feel of a fabric. With proper chemical treatment and selection of fabric construction, an undergarment of the present invention can be produced that provide a host of benefits to the wearer, such as staying clean longer and faster drying.

Stain repellent finishes are effective in resisting soil; however, if soil does penetrate the finish, it may be difficult to remove. To counteract this, there is available a fluorochemical finish that not only repels stains, but also promotes release of that stain during washing. One such treatment method known in the art is Dams (U.S. Pat. No. 5,276,175) which is incorporated by reference, in toto, for its teachings of soil repel and release methods. The repellency of this soil releasing fluorochemical treatment method is useful in the undergarment of the present invention. Applying a stain release finish enhances the ability of a fabric to release stains during laundering. For a release finish, liquids may not bead up, but may at least partially soak into the fabric. Soil Release test (AATCC 130) is used for evaluation.

Another approach to soil release treatment of fabrics for use in the instant invention is the use of selected acrylic polymers. Unlike the soil release fluorochemical finish, the acrylic soil release agents give no soil repellency. However, good soil release properties are obtained. To achieve outstanding soil release and some repellency, a blend of the fluorochemical and the acrylic soil release chemistries are often blended together. See, for example Dams (U.S. Pat. No. 5,276,175).

In a preferred embodiment of the present invention, the chemical barrier layer may be a polymer such as “Resists Spills” from Nano-Tex™ or “Repels and Releases Stains” from Nano-Tex™ or a similar product from 3M or DuPont or a stain release product. Examples of such treatment methods are described in Soane (U.S. Pat. No. 6,472,476), Linford (U.S. Pat. No. 6,544,594) and Soane (U.S. Pat. No. 6,607,994) and are incorporated in toto by reference for their treatment methods for such fabrics. Nanotechnology with respect to fabric, describes material so minute in size that is incorporated into fabrics to give properties such as wrinkle resistance for cotton, without affecting the fabric feel, breathability, or strength. Nano-pel™ specifically, which is manufactured by Nano-Tex™, confers repellency to water and oil spills, while keeping the original texture (softness in touch) and breathability.

In another embodiment of the invention, Nano-Tex Resist Spills™ fabric is used. Nano-Tex Resist Spills™ fabric resists stains by the use of “whiskers” aligned by proprietary “spines” designed to repel liquids, which are attached to the fibers utilizing molecular “hooks”. These whiskers and hooks are very small (i.e., no more than 1/1000^{th} the size of a cotton fiber). See www.Nano-Tex.com. Contrary to conventional repellent technologies which are more like a coating which wash or wear away over time, Nano-Tex Resists Spills™ fabrics achieve durability without sacrificing the natural texture and breathability of the fabric. Nano-Tex Resists Spills™ fabric protection has been designed as a superior repellant product. While repellency and stain release are on opposite ends of the spectrum in chemical formulation, the nature of the repellency gives the ability to resist stains. Nano-Tex Resists Spills™ fabrics focus on maximum repellency, while balancing the need for release and maintaining the natural texture and breathability of the fabric. Nano-Tex™ has established the performance specifications for the fabric based around repellant benefits. This Nano-Tex™ fabric is a chemical enhancement, attached at the molecular level to transform the fibers and is a preferred method of fabric treatment for the undergarment of the present invention.

Because the undergarment uses fabric with either stain repel, stain repel and release, and/or stain release only properties that will prevent stains from adhering to the fabric or that will release stains that do adhere, no part of the underwear needs to be black to disguise stains. In fact, the undergarment can be made using a wide variety of colors, embellishments, designs and cuts.

Also, part or all of the outer covering, interior panel and/or border of the undergarment can be made from a stain repel and/or stain release fabric that feels similar to untreated fabric. No part of the undergarment needs to be plastic or laminated.

The undergarment is composed of stain repel, stain repel and release, or stain release only fabric referred to as “stain repel and/or release” fabric. In the preferred embodiment of the invention, an interior panel of stain repel and/or release fabric covers the crotch area of the undergarment. Preferably, this panel or an additional panel(s) of stain repel and/or release fabric covers the seat of the undergarment and can extend up the back either partially or all the way up the backside to the top of the back of the undergarment. The same or another panel of this fabric with stain repel and/or release properties can cover no part, some or all of the front side of the undergarment. If leakage occurs from a tampon or sanitary napkin onto the panel or any part of the undergarment, the fabric can be wiped clean if the stain is fresh, or simply washed in the washing machine with no need for pre-treatment of stains.

To prevent leakage from the undergarment onto apparel, the panel of stain repel and/or release fabric may also contain a side border, preferably in a ruffled fashion, made of such treated fabric that will provide additional protection against leakage and help to keep the sanitary napkin in place. The border may be facing the interior part of the crotch area (as shown in FIG. 6) or may face away from the interior part towards the outside of the undergarment. The border may also may be short and extend straight up from the fabric panel at a right angle to the fabric panel. In yet another embodiment, the undergarment may have two borders, one facing inside the crotch area of the undergarment and another facing the outside.

Finally, the undergarment can consist, in whole or in part, of fabric that has also been treated to be antimicrobial.

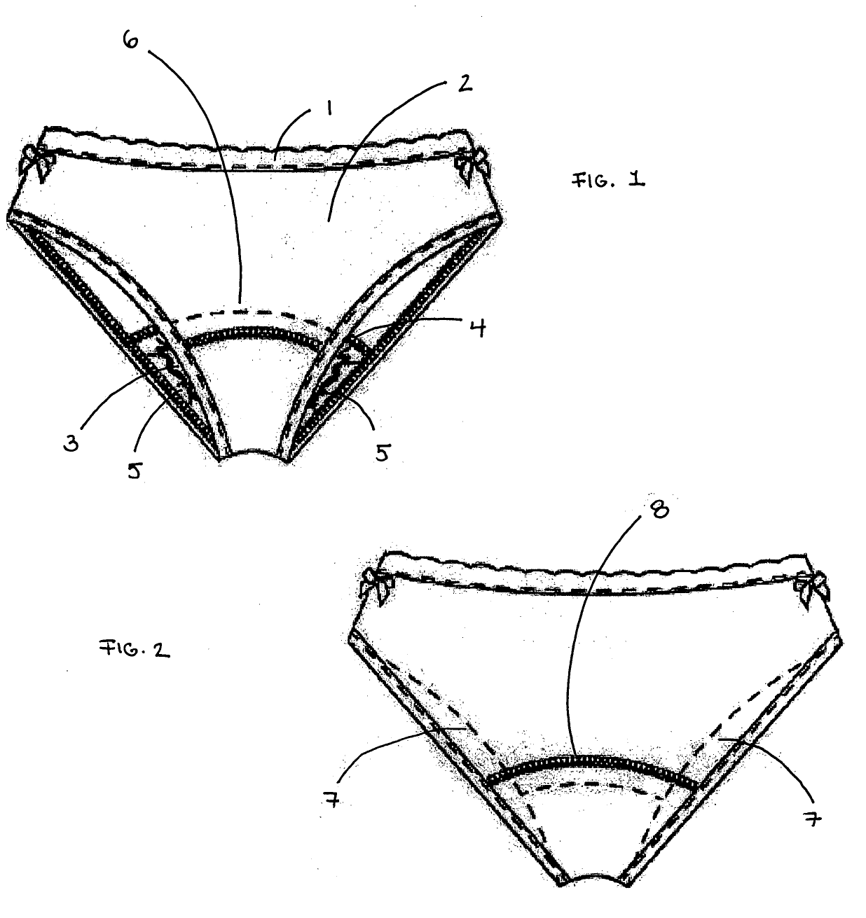

FIGS. 1 through 6 illustrate various embodiments of the invention.

FIG. 1 depicts a front view of a women's undergarment constructed according to one embodiment of the invention. An elastic waistband [1] is sewn around the waist opening in the outer layer [2] of stain repel and/or stain release material from which the panty is generally constructed.

In one embodiment of the invention, an interior panel made from stain repel and/or release fabric [3] is fully or partially sewn into the crotch area and extends partially up the backside of the undergarment [4]. A border made from stain repel and/or release fabric [5] is attached to or extends from the interior panel. A phantom outline [6] indicates how the interior panel may extend across the entire backside of the undergarment.

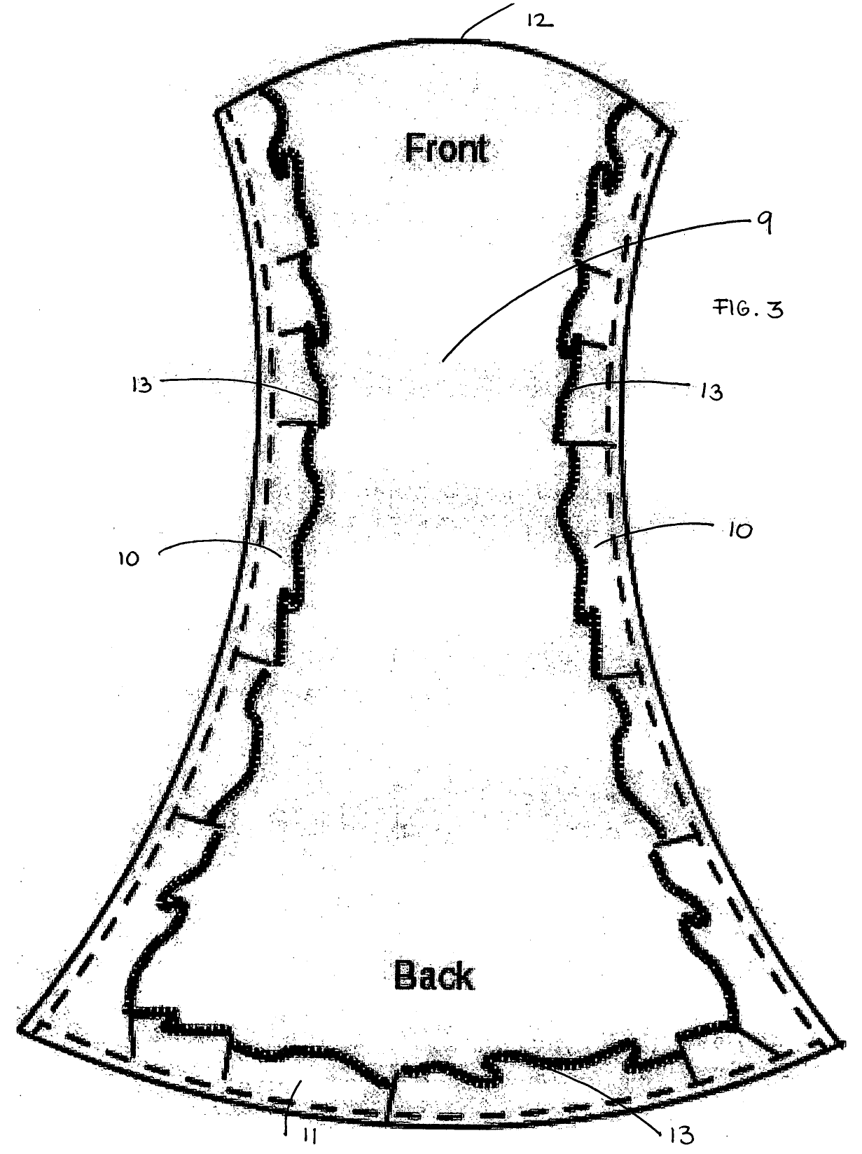

FIG. 2 depicts a back view of the same undergarment shown in FIG. 1. The edges of the front side of the undergarment are visible in phantom outline [7]. The interior panel made of stain repel and/or release fabric extends partially up the backside of the undergarment [8]. Following the outline of the interior panel, the border extends partially up the backside of the undergarment and over the top back of the panel to the other side. See FIG. 3 for a graphic representation of the interior panel that would be sewn, partially or fully into the panty.

FIG. 3 depicts one embodiment of an interior panel that provides partial coverage up the backside of the undergarment. The panel itself is composed of stain repel and/or release fabric [9]. The interior panel has additional stain repel and/or release fabric bordering the panel partially or fully. In this embodiment, the panel is bordered with ruffled stain repel and/or release fabric on the inside portions of the panel [10] and the back of the panel [11] but not in the front of the panel [12]. The border can either be partially or fully cut from the same piece of fabric as the interior panel or additional fabric stitched onto the interior panel. The border extends from the edges of the crotch area some distance into the crotch area, for example about 0.15-1.5 inches from the edges, preferably 0.5-1.5 centimeters from the edge. The extent to which the border extends into the crotch area may vary depending upon the manufacturer's preference, but the border is preferably large enough to catch fluids in that area of the panty, thereby preventing leakage. Alternatively the border may extend from the edges of the crotch area to some distance away from the crotch area; i.e., toward and into the leg opening. The border can be gathered or straight. In the illustrated embodiment of FIG. 3, the border is gathered in a ruffled fashion with a purl edging [13]. The border functions to provide additional protection from leakage of any liquids that may accumulate in the crotch area. It is understood that the ruffled border may be fashioned in any alternative manner, so that its function is maintained. In addition, it is contemplated within the scope of the invention that additional borders may be added and may face the inside crotch area or may face outward into the leg opening or both.

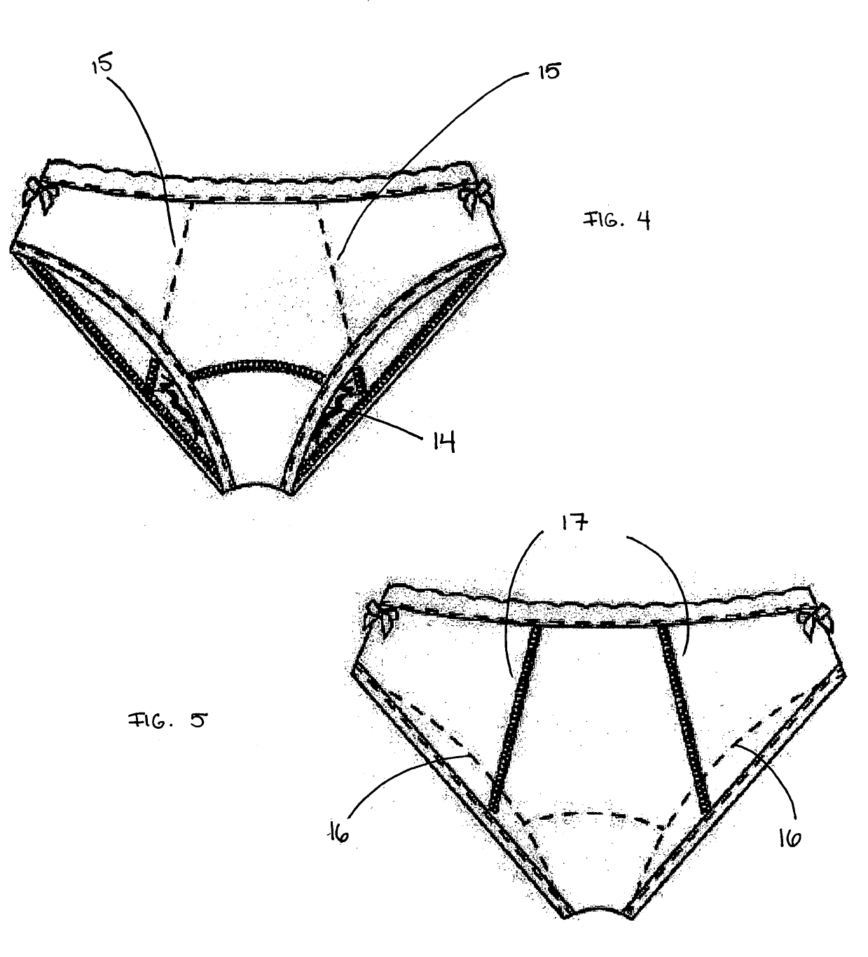

FIG. 4 depicts a front view of yet another embodiment of the undergarment constructed according to the invention. An interior panel made from stain repel and/or release fabric [14] may be sewn or attached to the crotch area and may extend fully up the backside of the undergarment. A phantom outline [15] indicates how the interior panel may extend up the entire backside of the undergarment to the elastic waist band.

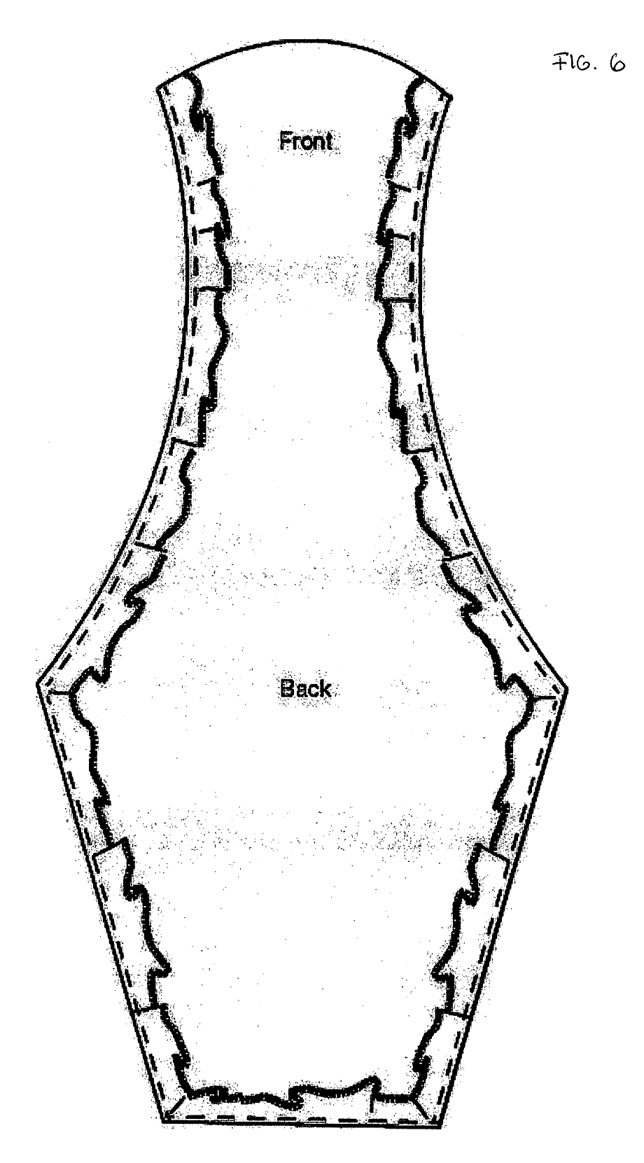

FIG. 5 depicts a back view of the same undergarment shown in FIG. 4. The edges of the front side of the undergarment are visible in phantom outline [16]. The interior panel made of stain repel and/or release fabric extends fully up the backside of the undergarment. In this embodiment, the interior panel is stitched in place [17].

FIG. 6 depicts a further embodiment of the interior panel that provides full coverage of the backside of the undergarment and has a border in a ruffled fashion that extends along the sides of the interior panel except for the front edge.

This undergarment may be worn with a conventional tampon and/or sanitary pad and is specifically designed to prevent staining of the undergarment itself and to prevent leakage and subsequent staining of apparel and bedding (when the wearer is sleeping). If the undergarment becomes soiled and the stain is fresh, the wearer can simply wipe the undergarment with a paper towel and continue to wear the undergarment until the wearer has the opportunity to change her undergarment. To wash the undergarment, the wearer only needs to wash it in the washing machine with no-pretreatment required.

The above description of various embodiments has been presented for purposes of illustration and description. It is not intended to be exhaustive or limiting to the precise forms disclosed. Obvious modifications or variations are possible in light of the above teachings. The embodiments discussed were chosen and described to provide illustrations and its practical application to thereby enable one of ordinary skill in the art to utilize the various embodiments and with various modifications as are suited to the particular use contemplated. All such modifications and variations are within the system as determined by the appended claims when interpreted in accordance with the breadth to which they are fairly, legally and equitably entitled.

I claim:

1. A lower-body undergarment comprising:
(a) the outer covering made from a fabric, treated with a chemical selected from the group consisting of: (i) stain repellant, (ii) stain repellant and releasable, or (iii) stain releasable, with a waist opening and leg openings;
(b) a fabric panel covering the crotch area made from a fabric, treated with a chemical selected from the group consisting of: (i) stain repellant, (ii) stain repellant and releasable, or (iii) stain releasable, such that said undergarment resists permanent stains; and
(c) said crotch area is lined with a border around the edges of the crotch area, which extends some distance from the edge of the crotch area, said border made from a fabric, treated with a chemical selected from the group consisting of: (i) stain repellant, (ii) stain repellant and releasable, or (iii) stain releasable, such that said undergarment protects against leakage of fluids.

2. The undergarment of claim 1 wherein the border extends some distance into the crotch area from the edge.

3. The undergarment of claim 1 wherein the border extends some distance away from the edge of the crotch area into the leg openings.

4. The undergarment of claim 1 comprising multiple borders around the edge of the crotch area.

5. The undergarment of claim 1 wherein said fabric panel or one or more additional fabric panels extend partially up the backside of the undergarment.

6. The undergarment of claim 1 wherein said fabric panel or one or more additional fabric panels extend fully up the backside of the undergarment.

7. The undergarment of claim 1 wherein said fabric panel or one or more additional fabric panels extend partially up the front side of the undergarment.

8. The undergarment of claim 1 wherein said fabric panel or one or more additional fabric panels extend fully up the front side of the undergarment.

9. The undergarment of claim 1 wherein said border is partially or fully cut from the same fabric as the fabric panel such that the border is created by folding over the fabric and stitching the fabric to create the border.

10. The undergarment of claim 1 wherein said fabric panel that covers the crotch area has a border that is partially or fully cut from separate fabric from the fabric panel such that the border is created by attaching one or more separate strips of fabric to the fabric panel.

11. The undergarment of claim 1 wherein said fabric panel that covers the crotch area has a border extending along the edge to help prevent leakage.

12. The undergarment of claim 1 wherein said fabric panel covering the crotch area has a border that is gathered, forming a ruffle.

13. The undergarment of claim 1 wherein said fabric panel covering the crotch area has a border wherein the border is flat.

14. The undergarment of claim 1 wherein the border is facing towards the middle of the fabric panel.

15. The undergarment of claim 1 wherein the border along the leg openings projects into the leg openings.

16. The undergarment of claim 1 wherein the border faces outwards away from the middle of the fabric panel.

17. The undergarment of claim 1 further comprising antibacterial/antimicrobial fabric.

18. The undergarment of claim 1 wherein the fabric is a treated cotton fabric.

19. The undergarment of claim 1 wherein the fabric is a treated cotton blend fabric.

20. The undergarment of claim 1 wherein the fabric is a treated bamboo fabric.

21. The undergarment of claim 1 wherein the fabric is a treated bamboo blend fabric.

22. The undergarment of claim 1 wherein the fabric is a treated synthetic fabric, such as acetate, nylon (polyamide), olefins (polypropylene, polyethylene), polyester, and/or spandex (including micro-fiber versions of the foregoing).

23. The undergarment of claim 1 wherein the fabric is a treated synthetic blend fabric.

24. The undergarment of claim 1 wherein the fabric is a treated silk fabric.

25. The undergarment of claim 1 wherein the fabric is a treated silk blend fabric.

26. The undergarment of claim 1 wherein the fabric is treated with fluorochemicals.

27. The undergarment of claim 1 wherein the fabric is a spill resistant nanotechnology.

28. The undergarment of claim 1 wherein the fabric is treated with acrylics.
